SEQUENCE LISTING (44 sequences)

SEQ ID NO: 1 - protein, 20 aa, Artificial Sequence
  Synthetic
EVAPGKRWFYNHVKQVPHLV

SEQ ID NO: 2 - protein, 23 aa, Artificial Sequence
  Synthetic
HEEVHMRPNKLSLTWAYTGPQLR

SEQ ID NO: 3 - protein, 13 aa, Artificial Sequence
  Synthetic
CTMPPSPRSKVIC

SEQ ID NO: 4 - protein, 13 aa, Artificial Sequence
  Synthetic
CTFPNTTMQRTFC

SEQ ID NO: 5 - protein, 13 aa, Artificial Sequence
  Synthetic
CTYPSWVAYIRFC

SEQ ID NO: 6 - protein, 13 aa, Artificial Sequence
  Synthetic
VCTYPPAHRTRFC

SEQ ID NO: 7 - protein, 13 aa, Artificial Sequence
  Synthetic
CTMPYHIHSIGLC

SEQ ID NO: 8 - protein, 13 aa, Artificial Sequence
  Synthetic
WCTIPSSMSIRLC

SEQ ID NO: 9 - protein, 13 aa, Artificial Sequence
  Synthetic
CHIGKRPVPCLWI

SEQ ID NO: 10 - protein, 13 aa, Artificial Sequence
  Synthetic
CYIGLRMVPCFHM

SEQ ID NO: 11 - protein, 13 aa, Artificial Sequence
  Synthetic
CTMPSHAVASFLC

SEQ ID NO: 12 - protein, 13 aa, Artificial Sequence
  Synthetic
CTMPVHTYSQWLC

SEQ ID NO: 13 - protein, 13 aa, Artificial Sequence
  Synthetic
CTYPPRFHMHWLC

SEQ ID NO: 14 - protein, 13 aa, Artificial Sequence
  Synthetic
CTHVAQWAIKAFC

SEQ ID NO: 15 - protein, 5 aa, Artificial Sequence
  Synthetic
PLGLA

SEQ ID NO: 16 - protein, 5 aa, Artificial Sequence
  Synthetic
SYGMH

SEQ ID NO: 17 - protein, 17 aa, Artificial Sequence
  Synthetic
VIWDDGSNKYYVDSVKG

SEQ ID NO: 18 - protein, 17 aa, Artificial Sequence
  Synthetic
DDYYGSGSFNSYYGTDV

SEQ ID NO: 19 - protein, 11 aa, Artificial Sequence
  Synthetic
RASQSVSIYLA

SEQ ID NO: 20 - protein, 7 aa, Artificial Sequence
  Synthetic
DASNRAT

SEQ ID NO: 21 - protein, 10 aa, Artificial Sequence
  Synthetic
QQRSNWPPFT

SEQ ID NO: 22 - protein, 126 aa, Artificial Sequence
  Synthetic
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 23 - protein, 108 aa, Artificial Sequence
  Synthetic
EIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIK

SEQ ID NO: 24 - protein, 8 aa, Artificial Sequence
  Synthetic
LSGRSDNH

SEQ ID NO: 25 - protein, 8 aa, Artificial Sequence
  Synthetic
TGRGPSWV

SEQ ID NO: 26 - protein, 15 aa, Artificial Sequence
  Synthetic
GSSGGSGGSGGSGGG

SEQ ID NO: 27 - protein, 5 aa, Artificial Sequence
  Synthetic
GGSSG

SEQ ID NO: 28 - protein, 5 aa, Artificial Sequence
  Synthetic
GGGGS

SEQ ID NO: 29 - protein, 215 aa, Artificial Sequence
  Synthetic
EIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 456 aa, Artificial Sequence
  Synthetic
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 501 aa, Artificial Sequence
  Synthetic
EVAPGKRWFYNHVKQVPHLVGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 504 aa, Artificial Sequence
  Synthetic
HEEVHMRPNKLSLTWAYTGPQLRGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 496 aa, Artificial Sequence
  Synthetic
QGCTMPPSPRSKVICGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 496 aa, Artificial Sequence
  Synthetic
QGCTFPNTTMQRTFCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 496 aa, Artificial Sequence
  Synthetic
QGCTYPSWVAYIRFCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 496 aa, Artificial Sequence
  Synthetic
QGVCTYPPAHRTRFCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 496 aa, Artificial Sequence
  Synthetic
QGCTMPYHIHSIGLCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 496 aa, Artificial Sequence
  Synthetic
QGWCTIPSSMSIRLCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 496 aa, Artificial Sequence
  Synthetic
QGCHIGKRPVPCLWIGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 496 aa, Artificial Sequence
  Synthetic
QGCYIGLRMVPCFHMGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 496 aa, Artificial Sequence
  Synthetic
QGCTMPSHAVASFLCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 496 aa, Artificial Sequence
  Synthetic
QGCTMPVHTYSQWLCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 496 aa, Artificial Sequence
  Synthetic
QGCTYPPRFHMHWLCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 496 aa, Artificial Sequence
  Synthetic
QGCTHVAQWAIKAFCGSSGGSGGSGGSGGGPLGLAGGSSGQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0181750 A1
(43) Pub. Date: Jun. 15, 2023

(54) MASK PEPTIDES AND MASKED ANTI-PTK7 ANTIBODIES COMPRISING SUCH

(71) Applicant: CRISPR Therapeutics AG, Zug (CH)

(72) Inventors: Jason Sagert, Cambridge, MA (US); Jui Dutta-Simmons, Cambridge, MA (US); Jonathan Alexander Terrett, Cambridge, MA (US)

(21) Appl. No.: 17/997,982

(22) PCT Filed: May 6, 2021
(86) PCT No.: PCT/IB2021/053849

§ 371 Date: Nov. 4, 2022

Related U.S. Application Data
(60) Provisional application No. 63/020,801, filed on May 6, 2020.

(51) Int. Cl. A61K 47/64; C07K 16/08; A61K 47/65

(52) U.S. Cl. CPC A61K 47/64; C07K 16/08; A61K 47/65; C07K 2317/92

(57) ABSTRACT

Mask peptides for use in producing masked antibodies specific to tyrosine-protein kinase-like 7 (PTK7). Also provided herein are masked anti-PTK7 antibodies comprising the mask peptide and therapeutic uses thereof.

### CROSS-REFERENCE TO RELATED APPLICATIONS

This application claims the benefit of the filing date of U.S. Provisional Application No. 63/020,801, filed May 6, 2020, the entire contents of which are incorporated by reference herein.

### BACKGROUND OF THE INVENTION

Protein tyrosine kinase 7 (PTK7), also known as colon carcinoma kinase 4 (CCK4), is a receptor protein tyrosine kinase that is involved in non-canonical Wnt signaling and comprises an extracellular domain. While PTK7 lacks detectable catalytic tyrosine kinase activity, it comprises signal transduction activity and is presumed to function in cellular adhesion. It is further thought that PTK7 is a marker for tumor progression in cancer, as it is expressed in various cancer cell lines, for example, colon and breast cancer cell lines.

### SUMMARY OF THE INVENTION

The present disclosure is based, at least in part, on the development of mask peptides that block binding of an anti-PTK7 antibody to the PTK7 antigen. Masked anti-PTK7 antibodies comprising such a mask peptide showed reduced binding activity to the PTK7 antigen and the binding activity was resumed upon removal of the mask peptide via protease cleavage. Such masked anti-PTK7 antibodies are expected to show promising anti-tumor effect with reduced toxicity.

Accordingly, one aspect of the present disclosure features a mask peptide, comprising (e.g., consisting of) the amino acid sequence of:

(a) EVAPGKRWFYNHVKQVPHLV (SEQ ID NO:1),

(b) HEEVHMRPNKLSLTWAYTGPQLR (SEQ ID NO:2), or

(c) X₁CX₂X₃X₄X₅X₆X₇X₈X₉X₁₀X₁₁X₁₂X₁₃, in which X₁ is V, W, or absent; X₂ is T, H, or Y; X₃ is M, F, Y, I, or H; X₄ is P, G, or V; X₅ is P, N, S, Y, K, L, V, or A; X₆ is S, T, W, A, H, R, or Q; X₇ is P, T, V, H, I, M, A, F, or W; X₈ R, M, A, H, V, Y, or absent; X₉ is S, Q, Y, T, P, A, M, or I; X₁₀ is K, R, I, C, S, Q, H, or absent; X₁₁ is V, T, R, L, F, W, or A; X₁₂ is I, F, L, W, or H; and X₁₃ is C, I, or M.

In some embodiments, the masked peptide may comprise (e.g., consists of) the amino acid sequence of (c), which can be:

(c1)
(SEQ ID NO: 3)
CTMPPSPRSKVIC,
(c2)
(SEQ ID NO: 4)
CTFPNTTMQRTFC,
(c3)
(SEQ ID NO: 5)
CTYPSWVAYIRFC,
(c4)
(SEQ ID NO: 6)
VCTYPPAHRTRFC,
(c5)
(SEQ ID NO: 7)
CTMPYHIHSIGLC,
(c6)
(SEQ ID NO: 8)
WCTIPSSMSIRLC,
(c7)
(SEQ ID NO: 9)
CHIGKRPVPCLWI,
(c8)
(SEQ ID NO: 10)
CYIGLRMVPCFHM,
(c9)
(SEQ ID NO: 11)
CTMPSHAVASFLC,
(c10)
(SEQ ID NO: 12)
CTMPVHTYSQWLC,
(c11)
(SEQ ID NO: 13)
CTYPPRFHMHWLC,
or
(c12)
(SEQ ID NO: 14)
CTHVAQWAIKAFC.

Any of the mask peptide disclosed herein may have 13-25 amino acids in length.

In another aspect, the masked antibody comprising an antibody that binds tyrosine-protein kinase-like 7 (PTK7) (anti-PTK7 antibody) and any of the mask peptides disclosed herein. Such a masked anti-PTK7 antibody comprises a heavy chain and a light chain, one of which can be linked to the mask peptide (e.g., at its N-terminus) via a protease cleavage site. The masked antibody has a reduced binding activity to the PTK7 relative to the anti-PTK7 antibody. In some examples, the mask peptide is linked to the N-terminus of the heavy chain of the anti-PTK7 antibody.

In some embodiments, the mask peptide is removable from the masked antibody by protease cleavage at the protease cleavage site. In some examples, the protease cleavage site can be a cleavage site of a matrix metalloproteinase (MMP), e.g., a motif of PLGLA (SEQ ID NO: 15).

In some embodiments, the mask peptide can be linked to the protease cleavage site via a first peptide linker. For example, the protease cleavage site can be linked to the N-terminus of the heavy chain or the light chain of the anti-PTK7 antibody via a second peptide linker. In some examples, the first peptide linker, the second peptide linker, or both are G/S peptide linkers. In some examples, the mask peptide is linked to the heavy chain of the anti-PTK7 antibody in a formula of: M-L₁-P-L₂-H, in which M represents the mask peptide, L₁ and L₂ represents the first and second peptide linkers, P represents the protease cleavage site, and H represents the heavy chain.

In some embodiments, the heavy chain of the anti-PTK7 antibody comprises a heavy chain variable domain (V_{H}), which comprises the same heavy chain complementary determining regions (CDRs) as the heavy chain CDRs of antibody Ab181; and/or wherein the anti-PTK7 antibody comprises a light chain variable domain (V_{L}), which comprises the same light chain complementary determining regions (CDRs) as the light chain CDRs of antibody Ab181. In some examples, the anti-PTK7 antibody comprises the same V_{H} as antibody Ab181 and/or the same V_{L} as antibody Ab181.

In some examples, the masked antibody comprises a heavy chain comprising an amino acid sequence selected from the group consisting of SEQ ID NOs: 30-44; and a light chain comprising the amino acid sequence of SEQ ID NO: 29.

Also within the scope of the present disclosure are a nucleic acid or a set of nucleic acids, which collectively encode any of the masked antibodies disclosed herein, a vector or a set of vectors (e.g., expression vectors), comprising the nucleic acid or the set of nucleic acids, and host cells comprising the vector or the set of vectors.

In addition, the present disclosure features a method for producing a masked antibody, comprising: (i) culturing a host cell comprising nucleic acids encoding any of the masked anti-PTK7 antibodies under conditions allowing for production of the masked-antibody encoded by the vector or set of vectors contained therein; and (ii) harvesting the host cell or the culture medium thereof for collecting the masked-antibody. In some embodiments, the method may further comprise isolating the masked-antibody from the host cell or the culture medium.

In yet another aspect, provided herein is a method for targeting PTK7-expressing cells in a tissue having presence of a protease, the method comprising administering to a subject in need thereof an effective amount of any of the masked anti-PTK7 antibodies disclosed herein. The mask peptide in the masked antibody can be removed by a protease at a tissue presenting the protease, which recognizes the protease cleavage site in the masked-antibody, thereby releasing the anti-PTK7 antibody to target PTK7-expressing cells in the tissue.

In some embodiments, the subject is a human cancer patient having or suspected of having a target disease, such as a cancer. Examples include non-small cell lung cancer, colon cancer, ovarian cancer, or breast cancer, which optionally is triple-negative breast cancer. In some examples, the masked anti-PTK7 antibody is conjugated to a diagnostic label. In other examples, the masked anti-PTK7 antibody is conjugated to a cytotoxic agent.

Further, the present disclosure provides a method for treating a cancer associated with PTK7-expressing cells, the method comprising administering to a subject in need thereof an effective amount of any of the masked anti-PTK7 antibodies as disclosed herein. The subject may be a human cancer patient having a cancer that comprises PTK7⁺ cancer cells and presents a protease that recognizes the protease cleavage site in the masked antibody. Exemplary cancers are provided herein. In some examples, the masked anti-PTK7 antibody can be conjugated to a cytotoxic agent.

Also within the scope of the present disclosure are (i) pharmaceutical compositions for use in treating or diagnosing a target disease (e.g., a target cancer as those disclosed herein), the pharmaceutical compositions comprising any of the masked anti-PTK7 antibodies and a pharmaceutically acceptable carrier; and (ii) uses of any of the masked anti-PTK7 antibodies disclosed herein for manufacturing a medicament for use in treating any of the target diseases as also disclosure here.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1B include data showing results from testing binding of anti-PTK7 probodies to PTK7 positive cells (SaOS-2 cells; osteosarcoma). FIG. 1A: a graph showing binding curves of anti-PTK7 probodies to PTK7 positive cells. FIG. 1B: a graph showing apparent K_{D} values of masked anti-PTK7 antibody.

FIGS. 2A-2G include data showing that masking peptides inhibit binding of anti-PTK7 antibodies to PTK7 positive cells (SaOS-2 cells; osteosarcoma). The binding inhibition of masked anti-PTK7 antibodies is disrupted by treatment of the masked antibody with MMP, the protease that cleaves the linker connecting the masking peptide to the antibody. FIGS. 2A-2F: graphs showing binding curves of masked anti-PTK7 antibodies to PTK7 positive cells in the absence and the presence of MMP14. FIG. 2G: a graph showing apparent KD of the indicated masked anti-PTK7 antibody in the absence and the presence of MMP14.

### DETAILED DESCRIPTION OF THE INVENTION

Multiple tumor-associated antigen targets have been progressed into clinical trials, chosen predominantly using the logic that expression in cancer tissues should be selective over normal tissues to avoid toxicity. PTK7 is reported to express on various of cancer cells and thus could serve as a potential tumor treatment target. However, excessive expression of PTK7 was also found in normal tissues, including lung, smooth muscle, stomach, kidney and bladder. Accordingly, there is a need to develop technology to reduce attack of normal tissues and cells in anti-PTK7-medicated tumor therapy.

The present disclosure is based, at least in part, on the development of mask peptides, which can inhibit (completely or partially) the binding of a masked anti-PTK7 antibody comprising such to the PTK7 antigen. The mask peptide is designed to be removable, for example, via protease cleavage, at a desired site (e.g., at a tumor site). Thus, the masked anti-PTK7 antibody has reduced or no binding activity to the PTK7 antigen until the masked peptide is removed at the desired site. Accordingly, the masked anti-PTK7 antibody would have low or no binding to normal cells and tissues, thereby addressing the potential toxicity concerns associated with conventional anti-PTK7 therapy.

Described herein are mask peptides and anti-PTK7 antibodies comprising such, nucleic acids encoding such, therapeutic applications of the masked anti-PTK7 antibodies, as well as methods for producing the masked anti-PTK7 antibodies.

### I. Mask Peptides

As used herein, a “mask peptide” for use in constructing a masked anti-PTK7 antibody can be a peptide capable of inhibiting, e.g., completely or partially, the binding of the antibody comprising such to the PTK7 antigen. For example, a mask peptide may reduce the binding activity of a masked anti-PTK7 antibody comprising such by at least 2-fold (e.g., at least 5-fold, at least 10-fold, at least 50-fold, at least 100-fold, at least 200-fold, at least 300-fold, at least 400-fold, at least 500-fold, at least 800-fold, at least 1,000-fold, at least 2,000-fold, at least 3,000-fold, at least 4,000-fold, or at least 5,000 fold) as compared with the same, unmasked anti-PTK7 antibody. In some embodiments, a mask peptide may substantially inhibit the binding activity of the masked anti-PTK7 antibody comprising such, leading to substantially no binding of the masked anti-PTK7 antibody to the PTK7 antigen, for example, undetectable binding by a conventional assay or very low binding that would be deemed biologically insignificant to those skilled in the art.

Any of the mask peptides disclosed herein may contain about 5-25 amino acid residues, for example, about 7-25 amino acid residues. In some examples, the mask peptides may have 13-25 amino acid residues in length, for example, 13, 14, 15, 16, 17, 18, 19, 20, 21, 22, 23, 24, or 25 amino acid residues in length. In some specific examples, the mask peptides disclosed herein may have 13 amino acid residues in length. In other specific examples, the mask peptides disclosed herein may have 20 amino acid residues in length. In yet other specific examples, the mask peptides disclosed herein may have 23 amino acid residues in length.

In some embodiments, the mask peptide disclosed herein may comprise the amino acid sequence of SEQ ID NO:1 or SEQ ID NO:2. See Table 2 below. In other embodiments, the mask peptide disclosed herein may comprise an amino acid sequence that share substantially homology to SEQ ID NO:1 or SEQ ID NO:2, for example, at least 80%, at least 85%, at least 90%, or at least 95% homology to SEQ ID NO:1 or SEQ ID NO:2.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some examples, the mask peptide disclosed herein may comprise an amino acid sequence having no more than 5 amino acid variations (e.g., containing 5, 4, 3, 2, or 1 amino acid variation) relative to SEQ ID NO: 1 or SEQ ID NO:2. In some instances, such amino acid variations can be amino acid residue substitutions, for example, conservative amino acid residue substitutions.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the mask peptide disclosed herein may comprise a motif of X₁CX₂X₃X₄X₅X₆X₇X₈X₉X₁₀X₁₁X₁₂X₁₃, in which X₁ is V, W, or absent; X₂ is T, H, or Y; X₃ is M, F, Y, I, or H; X₄ is P, G, or V; X₅ is P, N, S, Y, K, L, V, or A; X₆ is S, T, W, A, H, R, or Q; X₇ is P, T, V, H, I, M, A, F, or W; X₈ R, M, A, H, V, Y, or absent; X₉ is S, Q, Y, T, P, A, M, or I; X₁₀ is K, R, I, C, S, Q, H, or absent; X₁₁ is V, T, R, L, F, W, or A; X₁₂ is I, F, L, W, or H; and X₁₃ is C, I, or M.

In some examples, X₁ is V, W, or absent, X₂ is T, X₃ is M, F, Y, or I; X₄ is P; X₅ is P, N, S, Y, or V; X₆ is S, T, W, A, H, or R; X₇ is P, T, V, H, I, M, A, or F; X₈ R, M, A, H, V, Y, or absent; X₉ is S, Q, Y, T, A, or M; X₁₀ is K, R, I, S, Q, H, or absent; X₁₁ is V, T, R, F, or W; X₁₂ is I, F, or L; and X₁₃ is C.

In some embodiments, X₁ is absent; X₂ is H, or Y; X₃ is I; X₄ is G; X₅ is K, or L; X₆ is R; X₇ is P, or M; X₈ is V; X₉ is P; X₁₀ is C; X₁₁ is L, or F; X₁₂ is W or H; and X₁₃ is I, or M.

In some examples, the mask peptide may comprise the amino acid sequence of any one of SEQ ID NOs:3-14. In other examples, the mask peptide disclosed herein may comprise an amino acid sequence that share substantially homology to any one of SEQ ID NOs: 3-14, for example, at least 70%, at least 75%, at least 80%, at least 85%, or at least 90% homology to any one of SEQ ID NOs:3-14. Alternatively or in addition, the mask peptide disclosed herein may comprise an amino acid sequence having no more than 4 amino acid variations (e.g., containing 4, 3, 2, or 1 amino acid variation) relative to any one of SEQ ID NOs: 3-14. In some instances, such amino acid variations can be amino acid residue substitutions, for example, conservative amino acid residue substitutions.

In some examples, the mask peptide disclosed herein can be one of SEQ ID NOs:1-14. In some examples, the mask peptide can be a fragment of any one of SEQ ID NOs:1-14, which may have at least 5 consecutive amino acid residues (e.g., at least 6, at least 7, at least 8, at least 9, at least 10, or more).

In some instances, one or more amino acid residues can be added to the N-terminus of the mask peptide to maintain or improve stability of the peptide. In one example, the dipeptide QG can be added to the N-terminus of a mask peptide (e.g., a mask peptide comprising the amino acid sequence of one of SEQ ID NOs:3-14). Without being bound by theory, the Glutamine residue (particularly when it is located at the N-terminus) could spontaneously forms pyroglutamate, which helps protect the N-terminus against proteolysis.

### II. Masked Anti-PTK7 Antibodies

Also provided herein are masked anti-PTK7 antibodies that comprise any of the mask peptides disclosed herein. Relative to the unmasked counterpart, the masked anti-PTK7 antibodies disclosed herein have a reduced binding activity to the PTK7 antigen. For example, the binding activity may be reduced by at least 2-fold (e.g., at least 5-fold, at least 10-fold, at least 50-fold, at least 100-fold, at least 200-fold, at least 300-fold, at least 400-fold, at least 500-fold, at least 800-fold, at least 1,000-fold, at least 2,000-fold, at least 3,000-fold, at least 4,000-fold, or at least 5,000 fold). In some embodiments, a masked anti-PTK7 antibody disclosed herein may have undetectable binding activity to the PTK7 antigen or very low binding activity that would be deemed biologically insignificant to those skilled in the art as measured by a conventional assay.

(i) Anti-PTK7 Antibodies

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-PTK7 antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinforg.uk/abs).

The anti-PTK7 antibody described herein may be a full-length antibody, which contains two heavy chains and two light chains, each including a variable domain and a constant domain. Alternatively, the anti-PTK7 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human Such antibodies are non-naturally occurring, i.e., would not be produced in an animal without human act (e.g., immunizing such an animal with a desired antigen or fragment thereof or isolated from antibody libraries). Any of the antibodies described herein, e.g., anti-PTK7 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-PTK7 antibodies are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-PTK7 antibodies may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-PTK7 antibodies disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-PTK7 antibodies described herein specifically bind to the corresponding target antigen (e.g., PTK7) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (e.g., human PTK7) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some embodiments, an anti-PTK7 antibody as described herein has a suitable binding affinity for the target antigen (e.g., PTK7 such as human PTK7, see, e.g., Genbank accession no. AAH46109.1) or antigenic epitopes thereof. As used herein, “binding affinity” refers to the apparent association constant or K_{A}. The K_{A} is the reciprocal of the dissociation constant (K_{D}). The anti-PTK7 antibody described herein may have a binding affinity (K_{D}) of at least 100 nM, 10 nM, 1 nM, 0.1 nM, or lower for PTK7. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the anti-PTK antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-PTK7 antibody disclosed herein has an EC₅₀ value of lower than 10 nM, e.g., <1 nM, <0.5 nM, or lower than 0.1 nM, for binding to PTK7-positive cells. As used herein, EC₅₀ values refer to the minimum concentration of an antibody required to bind to 50% of the cells in a PTK7-positive cell population. EC₅₀ values can be determined using conventional assays and/or assays disclosed herein.

One exemplary anti-PTK7 antibody provided herein for making the masked anti-PTK7 antibodies is Ab181, the structural information of which is provided in Table 1 below.

TABLE 1
Exemplary Anti-PTK7 Antibody Ab181

| Component | Sequence | SEQ ID NO |
|---|---|---|
| Ab181 | SYGMH | 16 |
| VH CDR1 | | |
| Ab181 | VIWDDGSNKYYVDSVKG | 17 |
| VH CDR2 | | |
| Ab181 | DDYYGSGSFNSYYGTDV | 18 |
| VH CDR3 | | |
| Ab181 | RASQSVSIYLA | 19 |
| VL CDR1 | | |
| Ab181 | DASNRAT | 20 |
| VL CDR2 | | |
| Ab181 | QQRSNWPPFT | 21 |
| VL CDR3 | | |
| Ab181 V_{H} | QVQLVESGGGWQPGRSLRLSCAASGFTFSSYGMHWVRQAPGKGLE | 22 |
| CDR s-in bold | WVAVIWDDGSNKYYVDSVKGRFTISRDNSKNTLYLQMNSLRAEDTA | |
| | VYYCARDDYYGSGSFNSYYGTDVWGQGTTVTVSS | |
| Ab181 V_{L} | EIVLTQSPATLSLSPGERATLSCRASQSVSIYLAWYQQKPGQAPRL | 23 |
| CDRs-in | LIYDASNRATGIPARFSGSGSGTDFTLTISSLEPEDFAVYYCQQRS | |
| bold | NWPPFTFGPGTKVDIK | |

In some embodiments, the anti-PTK7 antibodies described herein may bind to the same epitope of a PTK7 polypeptide as antibody Ab181 or compete against Ab181 from binding to the PTK7 antigen. An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

In some embodiments, an anti-PTK7 antibody disclosed herein may comprise a heavy chain variable domain (V_{H}) having the same heavy chain complementary determining regions (CDRs) as those in Antibody Ab181 and/or a light chain variable domain (V_{L}) having the same light chain CDRs as those in Ab181. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinforg.uk/abs. bioinf.org.uk/abs/. The heavy chain and light chain CDRs of Ab181, and its V_{H} and V_{L} sequences are provided in Table 1 above.

In other embodiments, an anti-PTK7 antibody disclosed herein may be a functional variant of Ab181. Such a functional variant is substantially similar to Ab181, both structurally and functionally. A functional variant comprises substantially the same V_{H} and V_{L} CDRs as Ab181. For example, it may comprise only up to 8 (e.g., 8, 7, 6, 5, 4, 3, 2, or 1) amino acid residue variations in the total CDR regions relative to those in AB181 and binds the same epitope of PTK7 with substantially similar affinity (e.g., having a K_{D} value in the same order). In some instances, the functional variants may have the same heavy chain CDR3 as Ab181, and optionally the same light chain CDR3 as Ab181. Such an anti-PTK7 scFv may comprise a V_{H} fragment having CDR amino acid residue variations (e.g., up to 5, for example, 5, 4, 3, 2, and 1) in only the heavy chain CDR1 and/or CDR2 as compared with the V_{H} of Ab181. Alternatively or in addition, the anti-scFv antibody may further comprise a V_{L} fragment having CDR amino acid residue variations (e.g., up to 5, for example, 5, 4, 3, 2, and 1) in only the light chain CDR1 and/or CDR2 as compared with the V_{L} of Ab181. In some examples, the amino acid residue variations can be conservative amino acid residue substitutions.

In some embodiments, the anti-PTK7 antibody may comprise heavy chain CDRs that are at least 80% (e.g., 85%, 90%, 95%, or 98%) sequence identity, individually or collectively, as compared with the V_{H} CDRs of Ab181. Alternatively or in addition, the anti-PTK7 antibody may comprise light chain CDRs that are at least 80% (e.g., 85%, 90%, 95%, or 98%) sequence identity, individually or collectively, as compared with the V_{L} CDRs as Ab181. As used herein, “individually” means that one CDR of an antibody shares the indicated sequence identity relative to the corresponding CDR of the exemplary antibody. “Collectively” means that three V_{H} or V_{L} CDRs of an antiody in combination share the indicated sequence identity relative the corresponding three V_{H} or V_{L} CDRs of the exemplary antibody in combination.

Alternatively or in addition, the anti-PTK7 antibody disclosed herein may comprise a heavy chain variable region at least 70% (e.g., 80%, 85%, 90%, or 95%) identical to the V_{H} of Ab181, and/or a light chain variable region at least 70% (e.g., 80%, 85%, 90%, or 95%) identical to the V_{L} of Ab181.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the anti-PTK7 antibody disclosed herein may comprise the same V_{H} and/or same V_{L} chains as Ab181. See Table 1 above.

In some embodiments, the heavy chain of any of the anti-PTK7 antibodies as described herein (e.g., those derived from Ab181) may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-PTK antibody (e.g., those derived from Ab181) may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some embodiments, the anti-PTK7 antibody disclosed herein (e.g., those derived from Ab181) may be a single chain antibody (scFv). A scFv antibody may comprise a V_{H} fragment and a V_{L} fragment, which may be linked via a flexible peptide linker. In some instances, the scFv antibody may be in the V_{H}→V_{L} orientation (from N-terminus to C-terminus). In other instances, the scFv antibody may be in the V_{L}→V_{H} orientation (from N-terminus to C-terminus).

In some embodiments, the anti-PTK7 antibody as described herein, e.g., those derived from Ab181, can bind and inhibit (e.g., reduce or eliminate) the activity of PTK7-positive cells (e.g., tumor cells). In some embodiments, the anti-PTK7 antibody as described herein can bind and inhibit the activity of PTK7-positive cells by at least 30% (e.g., 35%, 40%, 45%, 50%, 60%, 70%, 80%, 90%, 95% or greater, including any increment therein). The inhibitory activity of an anti-PTK7 antibody described herein can be determined by routine methods known in the art, e.g., by an assay for measuring the K_{i,}^{app} value.

In some examples, the K_{i,}^{app} value of an antibody may be determined by measuring the inhibitory effect of different concentrations of the antibody on the extent of a relevant reaction; fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{( [ E ] - [ I ] - {K}_{i}^{app} ) + \sqrt{{( [ E ] - [ I ] - {K}_{i}^{app} )}^{2} + 4 [ E ] \cdot } {K}_{i}^{app}}{2} \qquad ( Equation 1 )$$

Where A is equivalent to vₒ/E, the initial velocity (v₀) of the enzymatic reaction in the absence of inhibitor (I) divided by the total enzyme concentration (E). In some embodiments, the anti-PTK7 antibody described herein may have a Ki^{app} value of 1000, 500, 100, 50, 40, 30, 20, 10, 5 pM or less for the target antigen or antigen epitope.

(ii) Masked Anti-PTK7 Antibody

The masked anti-PTK7 antibody disclosed here may comprise any of the anti-PTK antibodies disclosed herein and a mask peptide as also disclosed herein. The mask peptide may be linked to the N-terminus of either the heavy chain or the light chain of the antibody. In some embodiments, the mask peptide is linked to the N-terminus of the heavy chain, either directly or via a peptide linker. See disclosures herein.

A cleavage site such as a protease cleavage site can be located between the mask peptide and the heavy chain or light chain of the anti-PTK7 antibody. A cleavage site as used herein refers to a peptide motif, which can be cleaved under certain conditions, thereby separating its N-terminal fragment from its C-terminal fragment. By including a cleavage site between the mask peptide and the heavy or light chain of the antibody, the mask peptide can be removed at the cleavage site under the designed conditions, thereby releasing the fully functional anti-PTK7 antibody.

In some embodiments, the cleavage site is a protease cleavage site, where a protease cuts. Selection of a suitable protease cleavage site would depend on the desired action site of the anti-PTK7 antibody. For example, when a tumor site is the desired action site, a cleavage site of a protease specific to the tumor used for constructing a mask anti-PTK7 antibody intended to act at the tumor site. A protease specific to a tumor refers to any protease that has an elevated level and/or activity at the tumor site as relative to normal tissues.

In some examples, the protease cleavage site can be a cleavage site of a matrix metalloproteinase (MMP). In specific examples, the protease cleavage site can be a cleavage site of MMP14, for example, a motif of PLGLA (SEQ ID NO: 15). In other examples, the protease cleavage site can be a cleavage site for a serine or cysteine protease. In specific examples, the protease cleavage site can be a cleavage site for matriptase, e.g., a cleavage site having a motif of LSGRSDNH (SEQ ID NO: 24). In other specific examples, the protease cleavage site can be a cleavage site for urokinase-type plasminogen activator (uPA), e.g., a cleavage site having a motif of TGRGPSWV (SEQ ID NO: 25). Additional information regarding tumor-specific proteases and corresponding cleavage sites is known in the art, for example, disclosed in Vasiljeva et al., Scientific Reports, 10:5894, 2020, the relevant disclosures of which are incorporated by reference for the subject matter and purpose referenced herein.

Any of the mask peptides disclosed herein may be linked to the N-terminus of a protease cleavage site (e.g., those disclosed herein such as the MMP14 cleavage site). In some examples, the mask peptide is linked directly to the N-terminus of the protease cleavage site. In other examples, the mask peptide can be linked to the N-terminus of the protease cleavage site via a peptide linker. The protease cleavage site can be linked to the N-terminus of either the heavy chain or the light chain of the anti-PTK antibody as disclosed herein (e.g., linked to the heavy chain of the antibody). In some examples, the protease cleavage site can be linked directly to the N-terminus of the heavy or light chain. In other examples, the protease cleavage site can be linked to the N-terminus of the heavy or light chain via a peptide linker. In some examples, a same peptide linker may be used between the mask peptide and the protease cleavage site and between the protease cleavage site and the extracellular antigen binding domain. In other examples, different peptide linkers can be used.

In specific examples, a mask peptide as disclosed herein may be linked to the heavy chain of the anti-PTK antibody in a formula of M-L₁-P-L₂-H, in which M represents the mask peptide, L₁ and L₂ represents peptide linkers, P represents the protease cleavage site, and H represents the heavy chain. L₁ and L₂ may be identical in some instances. In other instances, L₁ and L₂ can be different.

In other specific examples, a mask peptide as disclosed herein may be linked to the light chain of the anti-PTK antibody in a formula of M-L₁-P-L₂-L, in which M represents the mask peptide, L₁ and L₂ represents peptide linkers, P represents the protease cleavage site, and L represents the heavy chain. L₁ and L₂ may be identical in some instances. In other instances, L₁ and L₂ can be different.

Any peptide linkers known in the art for use in linking two peptide or polypeptide fragments in a fusion polypeptide can be used in making the masked anti-PTK7 antibody disclosed herein. Such peptide linkers typically are enriched with flexible amino acid residues, for example, Gly and Ser (G/S rich linkers), so that the fragments flanking the linker can move freely relative to one another. The peptide linkers for use in the masked anti-PTK7 antibody may contain about 5-20 amino acid residues in length. When two linkers are used (L₁ and L₂ disclosed herein), the two linkers may be of the same length. Alternatively, they may have different lengths. Exemplary G/S rich linkers include, but are not limited to, GSSGGSGGSGGSGGG (SEQ ID NO: 26), GGSSG (SEQ ID NO: 27), a peptide containing one or multiple copies of GGGGS (SEQ ID NO: 28), or a peptide containing GS repeats.

Exemplary masked anti-PTK7 antibodies are provided in Table 4 below.

(iii) Preparation of Masked Anti-PTK7 Antibodies

Antibodies capable of binding PTK7 as described herein can be made by any method known in the art. See, for example, Harlow and Lane, (1998) Antibodies: A Laboratory Manual, Cold Spring Harbor Laboratory, New York. In some embodiments, the antibody may be produced by the conventional hybridoma technology. Alternatively, the anti-PTK7 antibody may be identified from a suitable library (e.g., a human antibody library).

Any of the masked anti-PTK7 antibodies as disclosed herein may be produced using the conventional recombinant technology as exemplified below.

Nucleic acids encoding the heavy and light chain of an antibody described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters (Brown, M. et al., Cell, 49:603-612 (1987)), those using the tetracycline repressor (tetR) (Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987)); Gossen and Bujard (1992); (M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(11):1811-1818, 1999). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an antibody described herein. The recombinant expression vector can be introduced into a suitable host cell (e.g., a dhfr− CHO cell) by a conventional method, e.g, calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of an antibody described herein (e.g., SEQ ID NOs: 30-44) and the other encoding the light chain of the antibody described herein (e.g., SEQ ID NO: 29). Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of any of the masked anti-PTK7 antibodies (e.g., those provided in Table 4 or variants thereof as disclosed herein), vectors (e.g., expression vectors) containing such, and host cells comprising the vectors are within the scope of the present disclosure. Also within the scope of the present disclosure are any method disclosed herein for producing the masked anti-PTK7 antibodies.

### III. Applications of Masked Anti-PTK7 Antibodies

Any of the masked anti-PTK7 antibodies disclosed herein can be used for therapeutic, diagnostic, and/or research purposes, all of which are within the scope of the present disclosure.

(i) Pharmaceutical Compositions

The masked anti-PTK7 antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease, e.g., a disease involving PTK7⁺ disease cells such as PTK7+ cancer cells. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The masked antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0. In some examples, the emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

(ii) Therapeutic Applications

Any of the masked anti-PTK7 antibodies disclosed herein may be used to inhibit and/or eliminate disease cells expressing PTK7, for example, PTK7⁺ cancer cells, thereby benefiting treatment of diseases and disorders involving such disease cells. In addition, any of the masked anti-PTK7 antibodies disclosed herein may be used to detect presence of PTK7+ cells at a disease site, for example, a tumor site.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human Mammals include, but are not limited to, farm animals, sport animals, pets, primates, horses, dogs, cats, mice and rats. A human subject who needs the treatment may be a human patient having, at risk for, or suspected of having a target disease/disorder characterized by carrying PTK7⁺ disease cells. Examples of such target diseases/disorders include cancer, for example, solid tumors.

In some embodiments, any of the masked antibodies as disclosed herein can be used for reducing or eliminating disease cells expressing PTK7 and thus treating diseases involving such disease cells. For example, the treatment method disclosed herein may be applied to patients (e.g., human patients) having a cancer, particularly a cancer that presents an elevated level of a protease (e.g., protein level or bioactivity level) relative to normal tissues. To treat such a cancer, the masked anti-PTK7 antibody that comprise a protease cleavage site recognizable by the protease presented at the cancer site can be used.

Non-limiting target cancer (e.g., solid tumors) include pancreatic cancer, gastric cancer, ovarian cancer, colon cancer, uterine cancer, breast cancer (e.g., triple-negative cancer), esophageal cancer, prostate cancer, testicular cancer, thyroid cancer, nasopharyngeal cancer, non-small cell lung (NSCLC), glioblastoma, neuronal, soft tissue sarcomas, melanoma. In other examples, the target cancer is leukemia, for example, Adult acute myeloid leukemia (AML).

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery. A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. In some examples, the dosage of the masked anti-PTK7 antibody described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of a masked antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history.

In some embodiments, more than one masked anti-PTK7 antibody, or a combination of a masked anti-PTK7 antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

In some embodiments, any of the masked anti-PTK7 antibodies disclosed herein may be conjugated to a cytotoxic agent (e.g., a small molecule cytotoxic agent capable of killing cells when delivered to the cells). Examples include, but are not limited to, alkylating agents (e.g., bendamustine, busulfan, carmustine, chlorambucil, cyclophosphamide, estramustine, ifosfamide, lomustine, melphalan, thiotepa, or treosulfan), anthracyclines (e.g., bleomycin, dactinomycin, daunorubicin, doxorubicin, epirubicin, idarubicin, mitomycin, or mitoxantrone) antimetabolites (e.g., azacitidine, capecitabine, cladribine, clofarabine, cytarabine, fludarabine, fluorouracil, gemcitabine, mercaptopurine, methotrexate, pemetrexed, raltitrexed, tioguanine), vinca alkaloid and etoposide (e.g., etoposide, vinblastine, vincristine, vindesine, or vinorelbine), protein kinase inhibitors (e.g., dasatinib, erlotinib, everolimus, imatinib, nilotinib, or sunitinib), or other antineoplastic agents (e.g., amsacrine, bexarotene, bortezomib, carboplatin, cetuximab, cisplatin, crisantaspase, dacarbazine, docetaxel, hydroxycarbamide (hydroxyurea), irinotecan, oxaliplatin, paclitaxel, pentostatin, procarbazine, temozolomide, topotecan, trastuzumab, or tretinoin)

In some embodiments, any of the masked anti-PTK7 antibodies disclosed herein may be conjugated to a detectable label. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

### IV. Kit for Use of Masked Anti-PTK7 Antibody

The present disclosure also provides kits for use in treating or alleviating a target disease or diagnosing a disease site such as any of the target cancers as described herein. Such kits can include one or more containers comprising a masked anti-PTK7 antibody, e.g., any of those described herein. In some instances, the masked anti-PTK7 antibody may be co-used with a second therapeutic agent.

In some embodiments, the kit can comprise instructions for use in accordance with any of the methods described herein. The included instructions can comprise a description of administration of the masked anti-PTK7 antibody, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of a masked anti-PTK7 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a masked anti-PTK7 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Identification of Masking Peptides Specific for Anti-PTK7 Antibody Ab181

This example describes the identification of masking peptides capable of blocking anti-PTK7 antibody Ab181 from binding to the PTK7 antigen using two different phage display screen assays. Screen 1 used a peptide library format of X₁₅/X₁₉ peptides. Screen 2 used a peptide library format of XₙCXₙCXₙ peptides. Both Screen 1 and Screen 2 used a series of rounds of selection with increasing stringency to identify specific peptide binders.

Screen 1 generated 2 unique peptide sequences (Table 2) for testing as masking peptides for antibodies. Screen 2 yielded a total of 27 unique peptide sequences, from which 12 unique peptide sequences (Table 3) were selected for testing based on levels of sequence enrichment and performance in the validation assays after screening.

TABLE 2
Unique peptide sequences that mask the binding
domain of Ab181 identified in Screen 1.

| | | SEQ ID |
|---|---|---|
| Mask name | Sequence | NO: |
| P1 | EVAPGKRWFYNHVKQVPHLV | 1 |
| P2 | HEEVHMRPNKLSLTWAYTGPQLR | 2 |

TABLE 3
Unique peptide sequences that mask the binding domain of Ab181
identified in Screen 2.

| Mask name | Family | Selected shortlist | Sequence | SEQ ID NO: |
|---|---|---|---|---|
| M3 | 1 | CSP_R2_38 | CTMPPSPRSKVIC | 3 |
| M4 | 1 | CSP_R2_29 | CTFPNTTMQRTFC | 4 |
| M5 | 1 | CSP_R2_10 | CTYPSWVAYIRFC | 5 |
| M6 | 1 | CSP_R2_2 | VCTYPPAHRTRFC | 6 |
| M7 | 1 | CSP_R2_28 | CTMPYHIHSIGLC | 7 |
| M8 | 1 | CSP_R2_19 | WCTIPSSMSIRLC | 8 |
| M9 | 2 | CSP_R2_22 | CHIGKRPVPCLWI | 9 |
| M10 | 2 | CSP_R2_39 | CYIGLRMVPCFHM | 10 |
| M11 | 1 | CSP_R3P2_25 | CTMPSHAVASFLC | 11 |
| M12 | 1 | CSP_R3P3_53 | CTMPVHTYSQWLC | 12 |
| M13 | 1 | CSP_R3P2_26 | CTYPPRFHMHWLC | 13 |
| M14 | 3 | CSP_R2_25 | CTHVAQWAIKAFC | 14 |

### Example 2: Engineering Masked Antibodies

Masked antibodies were designed using the sequences identified in the phage display library screens described in Example 1. For masked antibody constructs, the masking peptide was added to the Ab181 IgG1kappa heavy chain (HC) by a flexible linker sequence that also contained the substrate sequence (PLGLA; SEQ ID NO: 15) for Matrix Metalloproteinase (MMP) cleavage (Table 4).

TABLE 4
Masked antibody sequences.

| Antibody | Heavy chain Sequence | Light chain sequence |
|---|---|---|
| Ab181 | QVQLVESGGGVVQPGRSLRLSCAASGFTFS [symbol] | EIVLTQSPATLSLSPGERATLSC [symbol] |
| | [symbol] WVRQAPGKGLEWVA [symbol] | [symbol] WYQQKPGQAPRLLIYGIPA [symbol] |
| | [symbol] SRFTISRDNSKNTLYLQMNSLRAEDTAV | RFSGSGSGTDFTLTISSLEPEDFAVYYC [symbol] |
| | YYCAR [symbol] WGQGTTVTV | [symbol] TFGPGTKVDIKRTVAAPSVFIFP |
| | SSASTKGPSVFPLAPSSKSTSGGTAALGCLV | PSDEQLKSGTASVVCLLNNFYPREAKVQWK |
| | KDYFPEPVTVSWNSGALTSGVHTFPAVLQSS | VDNALQSGNSQESVTEQDSKDSTYSLSSTL |
| | GLYSLSSVVTVPSSSLGTQTYICNVNHKPSN | TLSKADYEKHKVYACEVTHQGLSSPVTKSF |
| | TKVDKKVEPKSCDKTHTCPPCPAPELLGGPS | NRGEC (SEQ ID NO: 29) |
| | VFLFPPKPKDTLMISRTPEVTCVVVDVSHED | |
| | PEVKFNWYVDGVEVHNAKTKPREEQYNSTYR | |
| | VVSVLTVLHQDWLNGKEYKCKVSNKALPAPI | |
| | EKTISKAKGQPREPQVYTLPPSRDELTKNQV | |
| | SLTCLVKGFYPSDIAVEWESNGQPENNYKTT | |
| | PPVLDSDGSFFLYSKLTVDKSRWQQGNVFSC | |
| | SVMHEALHNHYTQKSLSLSPGK (SEQ ID | |
| | NO: 30) | |
| Ab181.P1 | EVAPGKRWFYNHVKQVPHLVGSSGGSGGSGGSGG | Same as Ab181 |
| | GPLGLAGGSSGQVQLVESGGGVVQPGRSLRLSCA | |
| | ASGFTFSSYGMHWVRQAPGKGLEWVAVIWDDGSN | |
| | KYYVDSVKGRFTISRDNSKNTLYLQMNSLRAEDT | |
| | AVYYCARDDYYGSGSFNSYYGTDVWGQGTTVTVS | |
| | SASTKGPSVFPLAPSSKSTSGGTAALGCLVKDYF | |
| | PEPVTVSWNSGALTSGVHTFPAVLQSSGLYSLSS | |
| | VVTVPSSSLGTQTYICNVNHKPSNTKVDKKVEPK | |
| | SCDKTHTCPPCPAPELLGGPSVFLFPPKPKDTLM | |
| | ISRTPEVTCVVVDVSHEDPEVKFNWYVDGVEVHN | |
| | AKTKPREEQYNSTYRVVSVLTVLHQDWLNGKEYK | |
| | CKVSNKALPAPIEKTISKAKGQPREPQVYTLPPS | |
| | RDELTKNQVSLTCLVKGFYPSDIAVEWESNGQPE | |
| | NNYKTTPPVLDSDGSFFLYSKLTVDKSRWQQGNV | |
| | FSCSVMHEALHNHYTQKSLSLSPGK | |
| | (SEQ ID NO: 31) | |
| Ab181.P2 | HEEVHMRPNKLSLTWAYTGPQLRGSSGGSGGSGG | Same as Ab181 |
| | SGGGPLGLAGGSSGQVQLVESGGGVVQPGRSLRL | |
| | SCAASGFTFSSYGMHWVRQAPGKGLEWVAVIWDD | |
| | GSNKYYVDSVKGRFTISRDNSKNTLYLQMNSLRA | |
| | EDTAVYYCARDDYYGSGSFNSYYGTDVWGQGTTV | |
| | TVSSASTKGPSVFPLAPSSKSTSGGTAALGCLVK | |
| | DYFPEPVTVSWNSGALTSGVHTFPAVLQSSGLYS | |
| | LSSVVTVPSSSLGTQTYICNVNHKPSNTKVDKKV | |
| | EPKSCDKTHTCPPCPAPELLGGPSVFLFPPKPKD | |
| | TLMISRTPEVTCVVVDVSHEDPEVKFNWYVDGVE | |
| | VHNAKTKPREEQYNSTYRVVSVLTVLHQDWLNGK | |
| | EYKCKVSNKALPAPIEKTISKAKGQPREPQVYTL | |
| | PPSRDELTKNQVSLTCLVKGFYPSDIAVEWESNG | |
| | QPENNYKTTPPVLDSDGSFFLYSKLTVDKSRWQQ | |
| | GNVFSCSVMHEALHNHYTQKSLSLSPGK | |
| | (SEQ ID NO: 32) | |
| Ab181.M3 | QGCTMPPSPRSKVICGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 33) | |
| Ab181.M4 | QGCTFPNTTMQRTFCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 34) | |
| Ab181.M5 | QGCTYPSWVAYIRFCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 35) | |
| Ab181.M6 | QGVCTYPPAHRTRFCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 36) | |
| Ab181.M7 | QGCTMPYHIHSIGLCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 37) | |
| Ab181.M8 | QGWCTIPSSMSIRLCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 38) | |
| Ab181.M9 | QGCHIGKRPVPCLWIGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 39) | |
| Ab181.M10 | QGCYIGLRMVPCFHMGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 40) | |
| Ab181.M11 | QGCTMPSHAVASFLCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 41) | |
| Ab181.M12 | QGCTMPVHTYSQWLCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 42) | |
| Ab181.M13 | QGCTYPPRFHMHWLCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAKTKP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 43) | |
| Ab181.M14 | QG CTHVAQWAIKAFCGSSGGSGGSGGSGGGPLGL | Same as Ab181 |
| | AGGSSGQVQLVESGGGVVQPGRSLRLSCAASGFT | |
| | FSSYGMHWVRQAPGKGLEWVAVIWDDGSNKYYVD | |
| | SVKGRFTISRDNSKNTLYLQMNSLRAEDTAVYYC | |
| | ARDDYYGSGSFNSYYGTDVWGQGTTVTVSSASTK | |
| | GPSVFPLAPSSKSTSGGTAALGCLVKDYFPEPVT | |
| | VSWNSGALTSGVHTFPAVLQSSGLYSLSSVVTVP | |
| | SSSLGTQTYICNVNHKPSNTKVDKKVEPKSCDKT | |
| | HTCPPCPAPELLGGPSVFLFPPKPKDTLMISRTP | |
| | REEQYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| | (SEQ ID NO: 44) | |

The mask peptide in each of the masked antibody is in boldface and underlined. The heavy chain and light chain complementary determining regions in the parent Ab181 (following the Kabat numbering scheme) are boldfaced and italicized. See also Table 1 above.

### Example 3: Determining Binding Affinities of Masked Anti-PTK7 Antibodies and Reversal of Binding Inhibition by MMP14 Treatment

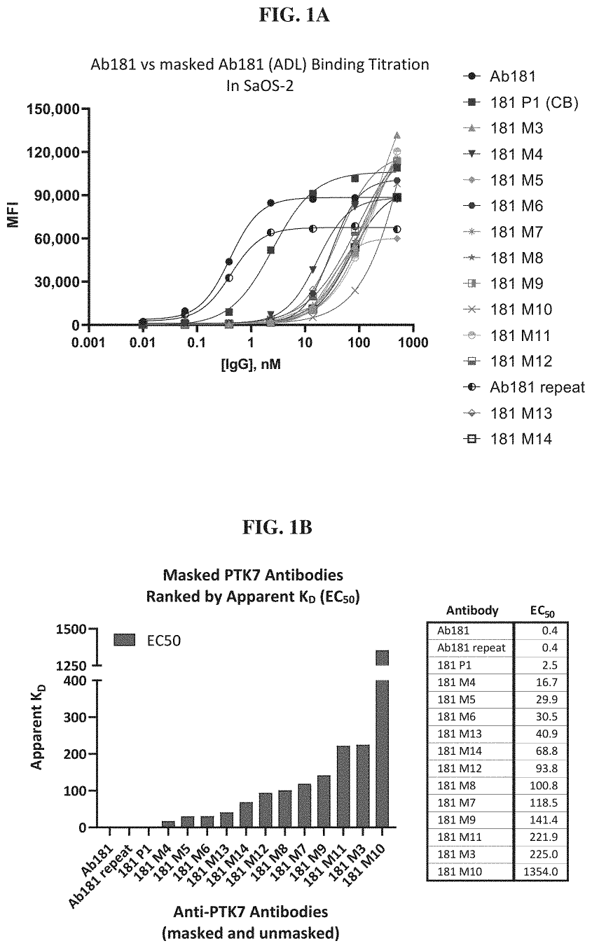

To test the ability of the different masking peptides to mask and/or inhibit binding of the anti-PTK7 antibody, binding titration assay were performed on PTK7 positive cell line SaOS-2 (osteosarcoma). Cells were plated (0.2×10⁶ cells/per well) and incubated with a dose titration (500 nM to 0.011 nM) of the antibodies listed in Table 4. Cells were incubated with antibodies for 30 minutes at 4° C. followed by washing and incubation with a universal secondary antibody, mouse anti-human Fc conjugated to APC (Biolegend cat #409305) for another 30 minutes at 4° C. Following washing, cells were fixed in a fixation buffer (IC Fixation Buffer, eBioscience cat #00-8222-49) at a 1:1 ratio, total volume of 200 μL, and run on a flow cytometer (Novocyte), collecting 10,000 events per well. Percent positive cells were calculated according to baselines set by no antibody controls (0 nM), and geometric Mean Fluorescent Intensities (MFI) of the total singlet cell populations were used to establish binding titration curves using 4-parameter nonlinear regression formula (Prism Graphpad) (FIG. 1A). Apparent EC₅₀ values were calculated from binding curves, and masking peptides were ranked in order of lowest to highest EC₅₀ as shown in FIG. 1B.

These results demonstrate the range of binding affinities of the different masking peptides in masked antibody format, which enables the masking peptides in masked antibody format to be used to inhibit binding of Ab181 to antigen PTK7 on target cells.

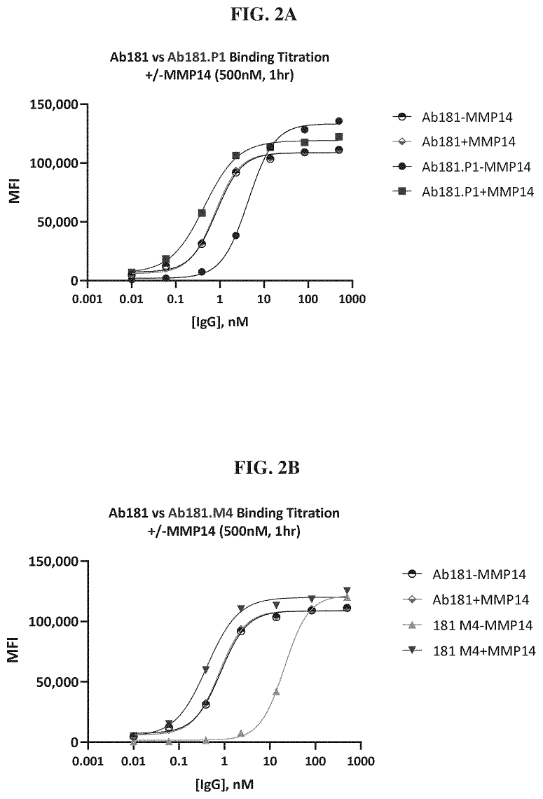

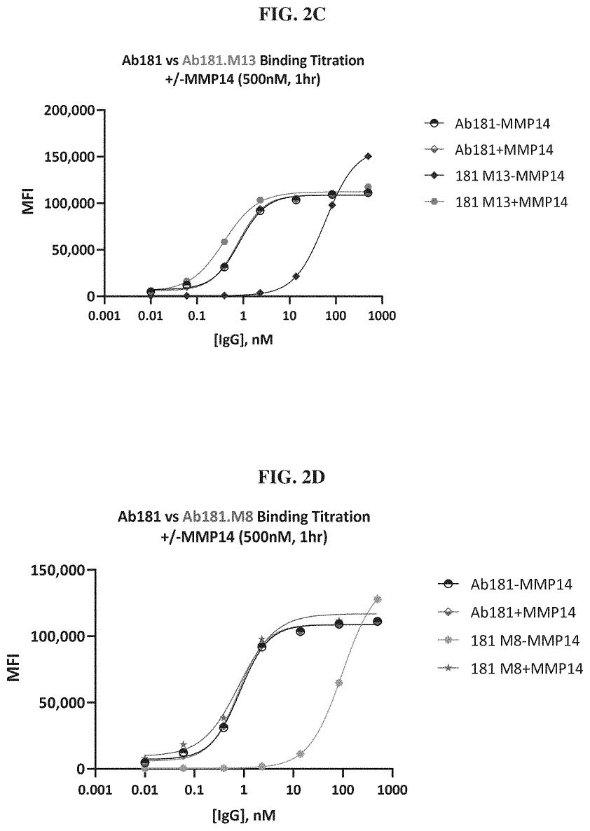

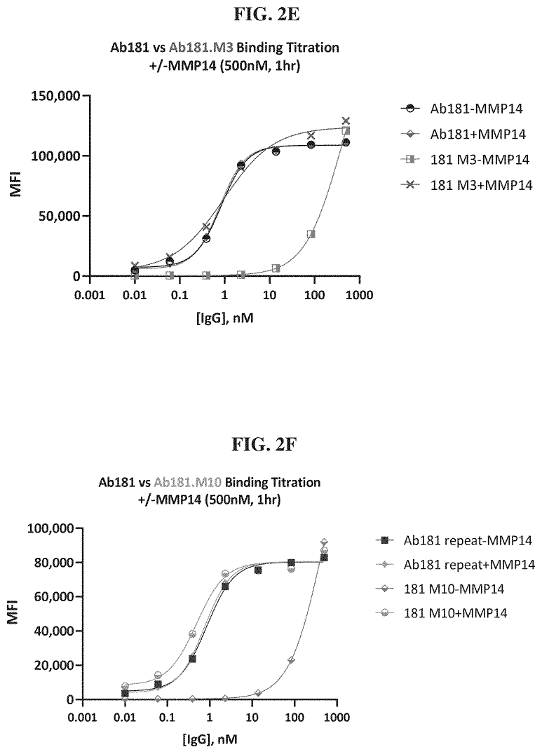

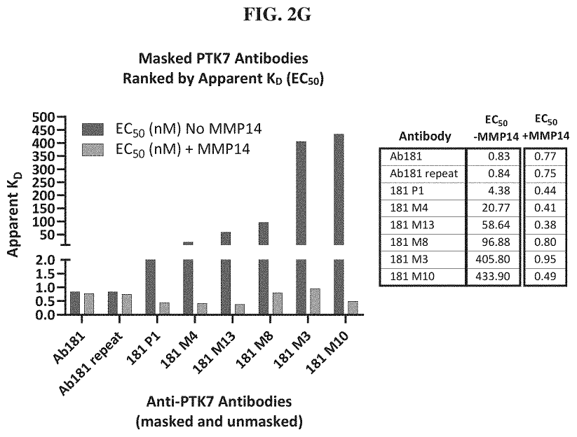

In addition to assessing the strength of the masking peptides for inhibiting antibody binding to PTK7 on the cell surface, it was demonstrated that this inhibition was reversible when the masked antibody was treated with MMP, the protease that cleaves the linker connecting the masking peptide to the antibody. MMP2, MMP 9 and MMP 14 were tested for the ability to cleave this sequence using in vitro binding assays. MMP14 provided more robust cleavage than MMP2 and MMP9, and therefore mask reversal studies were performed using MMP14. A subset of masked antibodies (6) were selected from the 14 candidates based on binding curve profile, sequence similarity, and apparent EC₅₀. Masked and unmasked antibodies were each incubated with 500 nM MMP14 (Enzo Biosciences Cat #ALX-201-098-0010) for 1 hour at room temperature before being used in a dose titration assay, alongside untreated antibodies, as described in Example 1. Binding titration curves (FIGS. 2A-2E) and apparent EC₅₀ values (FIG. 2G) were calculated as described in Example 1.

These results demonstrated that MMP14 treatment of the probodies led to effective reversal of their masking effect, regardless of the strength of the masking peptide, thereby highlighting that the masking peptide activity can be regulated.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A mask peptide, comprising the amino acid sequence selected from the group consisting of:
(a) EVAPGKRWFYNHVKQVPHLV (SEQ ID NO:1),
(b) HEEVHMRPNKLSLTWAYTGPQLR (SEQ ID NO:2), and
(c) X₁CX₂X₃X₄X₅X₆X₇X₈X₉X₁₀X₁₁X₁₂X₁₃, in which X₁ is V, W, or absent; X₂ is T, H, or Y; X₃ is M, F, Y, I, or H; X₄ is P, G, or V; X₅ is P, N, S, Y, K, L, V, or A; X₆ is S, T, W, A, H, R, or Q; X₇ is P, T, V, H, I, M, A, F, or W; X₈ R, M, A, H, V, Y, or absent; X₉ is S, Q, Y, T, P, A, M, or I; X₁₀ is K, R, I, C, S, Q, H, or absent; X₁₁ is V, T, R, L, F, W, or A; X₁₂ is I, F, L, W, or H; and X₁₃ is C, I, or M.

2. The mask peptide of claim 1, comprising the amino acid sequence of (c), which is:
(c1)
(SEQ ID NO: 3)
CTMPPSPRSKVIC,
(c2)
(SEQ ID NO: 4)
CTFPNTTMQRTFC,
(c3)
(SEQ ID NO: 5)
CTYPSWVAYIRFC,
(c4)
(SEQ ID NO: 6)
VCTYPPAHRTRFC,
(c5)
(SEQ ID NO: 7)
CTMPYHIHSIGLC,
(c6)
(SEQ ID NO: 8)
WCTIPSSMSIRLC,
(c7)
(SEQ ID NO: 9)
CHIGKRPVPCLWI,
(c8)
(SEQ ID NO: 10)
CYIGLRMVPCFHM,
(c9)
(SEQ ID NO: 11)
CTMPSHAVASFLC,
(c10)
(SEQ ID NO: 12)
CTMPVHTYSQWLC,
(c11)
(SEQ ID NO: 13)
CTYPPRFHMHWLC,
or
(c12)
(SEQ ID NO: 14)
CTHVAQWAIKAFC.

3. The mask peptide of claim 1, wherein the mask peptide is 13-25 amino acids in length.

4. The mask peptide of claim 1, wherein the mask peptide is:
(a)
(SEQ ID NO: 1)
EVAPGKRWFYNHVKQVPHLV,
(b)
(SEQ ID NO: 2)
HEEVHMRPNKLSLTWAYTGPQLR,
(c1)
(SEQ ID NO: 3)
CTMPPSPRSKVIC,
(c2)
(SEQ ID NO: 4)
CTFPNTTMQRTFC,
(c3)
(SEQ ID NO: 5)
CTYPSWVAYIRFC,
(c4)
(SEQ ID NO: 6)
VCTYPPAHRTRFC,
(c5)
(SEQ ID NO: 7)
CTMPYHIHSIGLC,
(c6)
(SEQ ID NO: 8)
WCTIPSSMSIRLC,
(c7)
(SEQ ID NO: 9)
CHIGKRPVPCLWI,
(c8)
(SEQ ID NO: 10)
CYIGLRMVPCFHM,
(c9)
(SEQ ID NO: 11)
CTMPSHAVASFLC,
(c10)
(SEQ ID NO: 12)
CTMPVHTYSQWLC,
(c11)
(SEQ ID NO: 13)
CTYPPRFHMHWLC,
or
(c12)
(SEQ ID NO: 14)
CTHVAQWAIKAFC.

5. A masked antibody comprising an antibody that binds tyrosine-protein kinase-like 7 (PTK7) (anti-PTK7 antibody) and a mask peptide of claim 1, wherein the anti-PTK7 antibody comprises a heavy chain and a light chain, one of which is linked to the mask peptide at its N-terminus via a protease cleavage site, and wherein the masked antibody has a reduced binding activity to the PTK7 relative to the anti-PTK7 antibody.

6. The masked antibody of claim 5, wherein the mask peptide is linked to the N-terminus of the heavy chain of the anti-PTK7 antibody.

7. The masked antibody of claim 5, wherein the mask peptide is removable by protease cleavage at the protease cleavage site.

8. The masked antibody of claim 5, wherein the protease cleavage site is a cleavage site of a matrix metalloproteinase (MMP).

9. The masked antibody of claim 8, wherein the protease cleavage site is a MMP14 cleavage site, which comprises the motif of PLGLA.

10. The masked antibody of claim 5, wherein the mask peptide is linked to the protease cleavage site via a first peptide linker.

11. The masked antibody of claim 5, wherein the protease cleavage site is linked to the N-terminus of the heavy chain or the light chain of the anti-PTK7 antibody via a second peptide linker.

12. The masked antibody of claim 10, wherein the first peptide linker, the second peptide linker, or both are G/S peptide linkers.

13. The masked antibody of claim 10, wherein the mask peptide is linked to the heavy chain of the anti-PTK7 antibody in a formula of: M-L₁-P-L₂-H, in which M represents the mask peptide, L₁ and L₂ represents the first and second peptide linkers, P represents the protease cleavage site, and H represents the heavy chain.

14. The masked antibody of claim 5, wherein the heavy chain of the anti-PTK7 antibody comprises a heavy chain variable domain (V_{H}), which comprises the same heavy chain complementary determining regions (CDRs) as the heavy chain CDRs of antibody Ab181; and/or wherein the anti-PTK7 antibody comprises a light chain variable domain (V_{L}), which comprises the same light chain complementary determining regions (CDRs) as the light chain CDRs of antibody Ab181.

15. The masked antibody of claim 14, wherein the anti-PTK7 antibody comprises the same V_{H} as antibody Ab181 and/or the same V_{L} as antibody Ab181.

16. The masked antibody of claim 15, wherein the masked antibody comprises a heavy chain comprising an amino acid sequence selected from the group consisting of SEQ ID NOs: 30-44; and a light chain comprising the amino acid sequence of SEQ ID NO: 29.

17. A nucleic acid or a set of nucleic acids, which collectively encode a masked antibody set forth in claim 1.

18. A vector or a set of vectors, comprising the nucleic acid or the set of nucleic acids of claim 17.

19. The vector or set of vectors of claim 18, which are expression vectors.

20. A host cell comprising the vector or set of vectors of claim 18.

21. A method for producing a masked antibody, comprising:
(i) culturing the host cell of claim 20 under conditions allowing for production of the masked-antibody encoded by the vector or set of vectors contained therein; and
(ii) harvesting the host cell or the culture medium thereof for collecting the masked-antibody.

22. The method of claim 21, further comprising isolating the masked-antibody from the host cell or the culture medium.

23. A method for targeting PTK7-expressing cells in a tissue having presence of a protease, the method comprising administering to a subject in need thereof an effective amount of a masked-antibody of claim 5, wherein the mask peptide in the masked antibody is removed by a protease at a tissue presenting the protease, which recognizes the protease cleavage site in the masked-antibody, thereby releasing the anti-PTK7 antibody to target PTK7-expressing cells in the tissue.

24. The method of claim 23, wherein the subject is a human cancer patient.

25. The method of claim 24, wherein the human cancer patient has non-small cell lung cancer, colon cancer, ovarian cancer, or breast cancer, which optionally is triple-negative breast cancer.

26. The method of claim 23, wherein the masked-antibody is conjugated to a diagnostic label or a cytotoxic agent.

27. A method for treating a cancer associated with PTK7-expressing cells, the method comprising administering to a subject in need thereof an effective amount of a masked-antibody of claim 5.

28. The method of claim 27, wherein the subject is a human cancer patient having a cancer that comprises PTK7⁺ cancer cells and presents a protease that recognizes the protease cleavage site in the masked antibody.

29. The method of claim 28, wherein the human cancer patient has non-small cell lung cancer, colon cancer, ovarian cancer, or breast cancer, which optionally is triple-negative breast cancer.

30. The method of claim 27, wherein the masked-antibody is conjugated to a cytotoxic agent.
